(19) United States
(12) Patent Application Publication
(10) Pub. No.: US 2019/0088152 A1
(43) Pub. Date: Mar. 21, 2019

(54) METHODS AND APPARATUS FOR REHABILITATION

(71) Applicants: New Jersey Institute of Technology, Newark, NJ (US); Rutgers, The State University of New Jersey, New Brunswick, NJ (US)

(72) Inventors: Sergei Adamovich, Garwood, NJ (US); Amanda Cronce, Hillsborough, NJ (US); Qinyin Qiu, Warren, NJ (US); Jigna Patel, Newark, NJ (US); Gerard Fluet, Newark, NJ (US); Alma Merians, Newark, NJ (US)

(73) Assignees: New Jersey Institute of Technology, Newark, NJ (US); Rutgers, The State University of New Jersey, New Brunswick, NJ (US)

(21) Appl. No.: 16/135,855

(22) Filed: Sep. 19, 2018

Related U.S. Application Data
(60) Provisional application No. 62/560,824, filed on Sep. 20, 2017.

(51) Int. Cl. G09B 5/06; A61B 5/11; A61B 5/00; A63F 13/25; A63F 13/80

(52) U.S. Cl. CPC G09B 5/06; A61B 5/1124; A61B 5/1128; A61B 2505/09; A63F 13/25; A63F 13/80; A61B 5/0077

(57) ABSTRACT

A rehabilitation system is disclosed herein. In some embodiments, the rehabilitation system comprises a first computer terminal accessible by a first user, the first computer terminal having a memory for storing instructions including a game for rehabilitation of at least one of the first user's shoulder, elbow, wrist, or fingers; a sensor for detecting motion of the first user's hand as the first user is playing the game; a second computer terminal that displays the game in real-time on the second computer terminal as the first user is playing the game on the first computer terminal, and wherein a difficulty level of the game can be remotely adjusted from the second computer terminal; and a server that acquires rehabilitation data from the first computer terminal as the first user plays the game, and wherein the server permits access, on the second computer terminal, to the rehabilitation data collected.

### CROSS-REFERENCE TO RELATED APPLICATION(S)

The present application claims priority to U.S. provisional patent application No. 62/560,824, filed on Sep. 20, 2017, which is incorporated herein by reference.

### STATEMENT REGARDING FEDERALLY SPONSORED RESEARCH

This invention was made with government support under Agreement No. 90RE5021 awarded by the National Institute on Disability, Independent Living, and Rehabilitation Research. The government has certain rights in the invention.

### FIELD OF THE DISCLOSURE

The present disclosure relates to methods and apparatus for rehabilitation. In particular, the present disclosure relates to a home based virtual reality system for rehabilitation of a patient's arm with focus on the hand.

### BACKGROUND

It is well known that complications due to overwork, injury and disease, such as stroke can all cause either total or partial hand injury or dysfunction. The loss of hand function significantly affects the quality of life by profoundly impacting a person's ability to perform daily self-care activities, such as feeding, dressing, and grooming. Additionally, the loss of hand function may limit the use of assistive technology aimed at improving quality of life. Although hand dexterity is important to restore full independence and reduce costly supportive care, few studies have addressed hand rehabilitation post stroke. Recovery of hand function post brain injury is particularly recalcitrant to currently available interventions.

When therapy is initially provided in a hospital or inpatient rehabilitation center, the patient is usually seen for two or three, half hour sessions per day. Often a neurological injury requires longer term therapy to restore the total functionality of the hand and arm, and outpatient or at home therapy is needed. An outpatient is generally seen two or three times a week for up to 6 months. It is well documented that typical outpatient rehabilitation is not provided at a volume or intensity necessary to induce the neural reorganization necessary for the recovery of motor function. In addition, since the patient has an injury, getting to the outpatient department for therapy is an issue.

At home rehabilitation therapies also have limitations, including lack of interaction by a health care provider. Some current state of the art technology utilizes Virtual Reality (VR) programs, such as games, to provide stimulation and continual repetition and intensity to strengthen relevant hand functional tasks. However, these current state of the art systems lack the ability to fully monitor all movements of the hand, even if the system utilizes a 3-dimensional (3-D) imaging device. Furthermore, these current systems don't provide the ability for a health professional to fully interact with the patient's rehabilitation therapy.

With current therapeutic delivery mechanisms, there still remains a need to provide greater intensity and extent of practice to induce neural reorganization. There is also a need for a system that fully monitors the movement of the hand while allowing full interaction by a health care provider to control, among other things, the intensity of the therapy to maintain high quality intensive physical therapy.

### SUMMARY

The present invention solves the problems of current state of the art systems and provides many more benefits beyond this. Methods and apparatus for rehabilitation of a patient's arm with focus on at least one of the patient's shoulder, elbow, wrist or fingers are disclosed herein. One aspect of the present disclosure relates to a rehabilitation system. The system includes a first computer terminal accessible by a first user. The first computer terminal has a memory for storing instructions and a processor to execute the instructions (the instructions including a game for rehabilitation of at least one of the first user's shoulder, elbow, wrist, or fingers). A sensor for detecting motion of the first user's hand, wrist, and fingers as the first user is playing the game is electrically coupled to the first computer terminal. The system also includes a second computer terminal accessible by a second user. The second computer terminal displays the game in real-time on the second computer terminal as the first user is playing the game on the first computer terminal. The game on the first computer terminal can be remotely adjusted from the second computer terminal. The system also includes a server electronically coupled to the first computer terminal and the second computer terminal. The server acquires real-time rehabilitation data from the first computer terminal as the first user plays the game. The server permits access, on the second computer terminal, to the rehabilitation data collected. The system further includes an external support for the patient's arm, wherein the support holds the patient's arm above the sensor and allows the patient to have free movement of the patient's wrist, hand, and fingers.

The disclosed system, method, and computer readable storage medium also solve certain internet based challenges not previously addressed in the healthcare industry. These challenges include expanding the capabilities of computerized hand therapy programs in the outpatient setting. The disclosed provides repurposing of existing computerized programs, such as computer games, through reprogramming, or even utilizing new computer programs, such as new computer games, through at least one processing engine for the use of hand therapy. This one processing engine is capable of running and storing the computer game that results in increased intensity of the game and/or deviation of the gaming program for purposes of patient therapy, and improves the speed and security of the computer based network therapy with control access of the program not only by the patient user, but with a third party non-user (healthcare provider or therapist) where the third party non-user may control the program based on medical necessity.

Furthermore, the disclosed embodiment utilizes unconventional software architecture and processes that are not routinely integrated in computerized hand therapy systems. Typical computer games used for therapy react only to the input of the user and may increase in intensity as the user demonstrates increased strength. The system utilizes an architecture that permits a third party non-user, such as a health care provider, to monitor patient progress and adjust the therapy program offsite of the actual therapy, regardless of the patient user input. The third party can modify the intensity of the program, for example increase or decrease the difficulty or intensity of the game, or even switch one game program to a different, different, more appropriate program. This unconventional architecture allows the healthcare provider to adjust the patient's therapy while the patient to utilize the therapy in the comfort of their own home. Thus, this home therapy system has the many of the advantage of that of an in hospital therapy session without the drawbacks of having the patient go to the hospital for an in-person therapy session.

The disclosed embodiment also utilizes at least one camera, such as, but not limited to, a charged coupled device (CCD) camera and/or a complementary metal oxide semiconductor (CMOS) camera, to fully capture motion of the patient's shoulder/elbow, wrist, and/or fingers, and translate that motion to the virtual environment of the game. This type of motion capturing method allows full 3-D imaging of the patient's hand and motion capture for analysis not provided in 2-D or other current 3-D systems.

The disclosed embodiment employs a non-routine system in which a first computer, or other computerized display device utilized by the patient user, and a second computer or other computerized display device interacts over an internet connected network or encrypted and stored connected centralized or decentralized server devices. Among the distinctions between the disclosed embodiment and known conventional methods is that the disclosed embodiment may operate under the total control of a third party that is a non-user to the therapy program. In addition, the third party may adjust the intensity of the therapy program to the user unrelated to what the user input in providing on the first computer. In addition, depending on the implementation, the program may be jointly controlled by both the user and the third party non user (healthcare professional). This non-routine system allows never seen before flexibility for the health care provider to treat a patient in an outpatient/at home and remote setting, which could not be achieved in the past using previously state of the art devices.

The above objectives are met by the present invention. Also, several additional advantages of the present invention will become apparent from the hereinafter-set forth Brief Description of the Drawings, Detailed Description, and claims appended herewith. These features and other features are described and shown in the following drawings and detailed description.

### BRIEF DESCRIPTION OF THE DRAWINGS

To assist those of skill in the art in creating and using a rehabilitation system and method, reference is made to the accompanying figures, wherein:

FIG. 1 is a schematic view of a rehabilitation system in accordance with some embodiments of the present disclosure.

FIG. 2 is a picture of a portion of a rehabilitation system in accordance with some embodiments of the present disclosure.

FIG. 3 is a screen view of a game used by a rehabilitation system in accordance with some embodiments of the present disclosure.

### DETAILED DESCRIPTION

Methods and apparatus for rehabilitation of a patient's arm with focus on the hand are disclosed herein. In some embodiments, the wrist and/or fingers are the focus of the rehabilitation. The patient may require rehabilitation in response to a brain injury, such as caused by external trauma, stroke, or the like, that has caused a loss of motor function. However, the present disclosure is not limited thereto, and patients that have had surgery for broken bones, damaged tendons and/or ligaments in the hand, wrist, and/or fingers, or for conditions, such as carpel tunnel syndrome and the like, may benefit from the methods and apparatus disclosed herein. Advantageously, the rehabilitation system disclosed herein may be used at-home and does not require cumbersome apparatus such as gloves with sensors embedded therein for monitoring progress during rehabilitation (a stroke victim, who has reduced motor function, would find difficulty putting on an apparatus, such as a glove, for rehabilitation without a therapist or home health aide present).

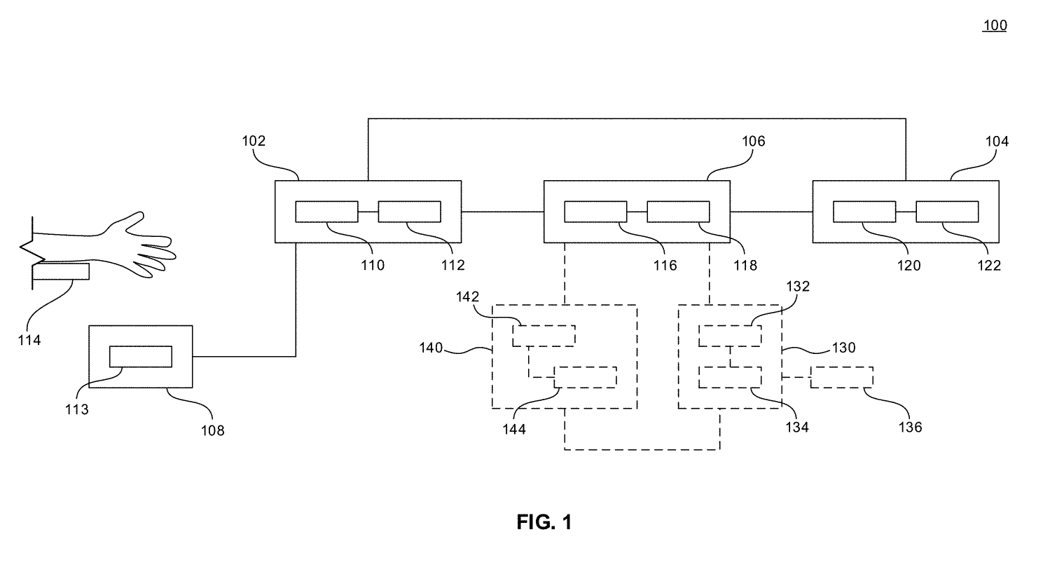

FIG. 1 is a schematic view of a rehabilitation system 100 in accordance with some embodiments of the present disclosure. The rehabilitations system 100 includes a first computer terminal 102 accessible by a first user, such as a patient, a second computer terminal 104 accessible by a second user, a server 106, and a sensor for detecting motion 108.

The first computer terminal 102 includes a memory 110 for storing instructions and a processor 112 for executing the instructions. The first computer terminal can be any number of various desktop or portable computer devices provided the computer devices can be electrically or physically coupled to the sensor 108. The instructions stored in the memory 110 include a game for rehabilitation of patient's hand, wrist, or fingers. Various exemplary embodiments of game which can be used for rehabilitation are discussed herein.

The sensor 108 can be electrically or physically coupled to the first computer terminal 102 via any suitable type of connection. Exemplary wireless connections include blue tooth or Wi-Fi. Exemplary physical connections include USB. The sensor 108 can detect motion of the hand, wrist, and fingers. The sensor 108 includes at least one infrared (IR) sensor 113. One exemplary commercially available motion tracking sensor is the Leap Motion Controller (LMC) by Leap Motion, Inc. The LMC is a computer hardware sensor that captures detailed hand movement as well as hand gestures and translates this information into virtual hand movement and object manipulation in the virtual environment. The LMC detects all hand motions and gestures within a limited space directly in front of the device. The LMC includes two cameras and three infrared LEDs. However, the sensor 108 can include any number of IR sensors and cameras suitable to capture motion of the patient's hand, wrist, and/or fingers, and translate that motion to the virtual environment of the game.

Data from the sensor 108 can be read into the memory 110 and the USB controller performs any necessary resolution adjustments. This data is then streamed via USB to an application program interface (API), such as the Leap Motion's API, a suite of programmable software that translates LMC data, into movement data which can be used to present any moving body in a virtually simulated movement. From the API, the system is programmed to feed tracking data into virtual reality activities by its API.

In addition to the first computer terminal 102 and the sensor 108, an at-home, e.g., the patient's home, portion of the system 100 may optionally include a support 114. In some embodiments, the support 114 can be absent if the patient has sufficient arm and/or shoulder strength to maintain the hand, wrist, and fingers in a raised position above the sensor 108. In other embodiments, where the patient does not have the sufficient strength, the support 114 can be provided to stabilize the patient's arm such that the hand, wrist and fingers can be is a raised position above the sensor 108. An exemplary commercially available support is the Armon™ Edero by Armon Products B.V. The support 114 can have numerous variations. Exemplary variations include a spring compensation system or a motorized system having one or more degrees of freedom (DOF). The support 114 can be mounted in various ways. For example, the support 114 can be mounted to the patient's wheel chair, to a table surface, to the arm of chair, and the like.

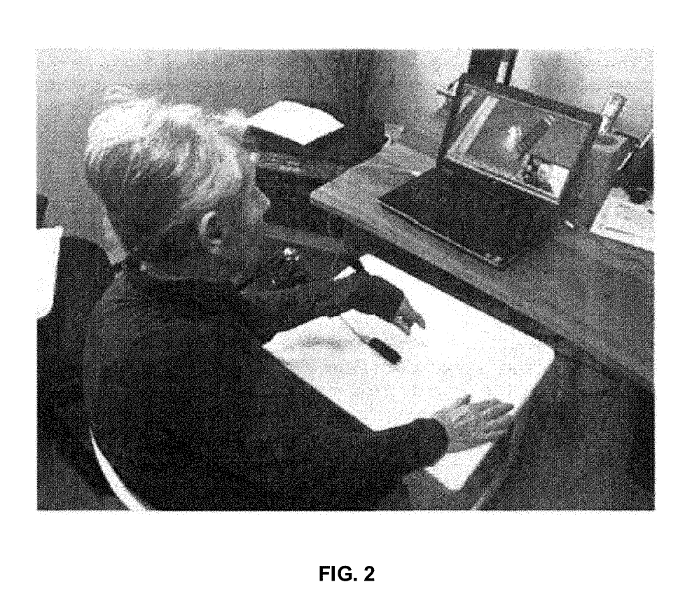

An exemplary embodiment of the ‘at-home’ portion of the system 100 is illustrated in FIG. 2. As illustrated, the patient's left arm is supported by a support which maintains the patient's left hand, wrist, and fingers above a sensor. The sensor is physical coupled to a laptop computer via a USB connection. The laptop computer displays a game that the patient is playing by making motions of the hand above the sensor.

The first computer terminal 102 is connected via a fiber optic or other type of communication line provided by an internet service provider (ISP) to the server 106. The server 106 includes a memory 116 and a processor 118. The motion data collected when the patient is playing the game can be downloaded to the server 106 and accessed on the server 106 by a medical professional.

The memory 116 may store instructions for one or more games for rehabilitation. The one or more games can be downloaded from the server 106 to the first computer terminal 102 by the patient if access to the server 106 is permitted from the first computer terminal 102.

The server 106 can collect data from more than one patient and can allow selectively access the data depending on the professional requesting access. For example, a healthcare professional, such as a therapist, who is working with a specific patient(s) can access the patient(s) performance data and adjust the difficulty level of the game based on their performance. In the case of healthcare researcher, game developer, and the like, the data collected from numerous patients may be averaged and provided to the researchers and game developers in a manner that does not identify personal information of each patient. In all cases, a healthcare professional, researcher, and/or game developer can access and download data from the server for which those individuals have been granted permission to view.

For example, and optionally, the rehabilitation system may further a third computer terminal 130 (which has the same components as the first computer terminal 102) accessible by a third user, the first computer terminal having a memory 132 for storing instructions and a processor 134 to execute the instructions, the instructions including a game for rehabilitation of the third user's hand. A second sensor 136 (which as the same components as the sensor 108), for detecting motion of the third user's hand, wrist, and fingers as the third user is playing the game, is electrically coupled to the third computer terminal 130. The third computer terminal is electronically coupled to the server 106, and the server acquires real-time rehabilitation data from the third computer terminal 130 as the third user plays the game.

The system may further include a fourth computer terminal 140 (which has the same components as the second computer terminal 104) accessible by a fourth user, wherein the fourth computer terminal displays the game in real-time on the third computer terminal as the third user is playing the game on the third computer terminal. A difficulty level of the game on the third computer terminal can be remotely adjusted from the fourth computer terminal. The server 106 acquires real-time rehabilitation data from the third computer terminal 130 as the third user plays the game, and wherein the server 106 permits access, on the fourth computer terminal 140, to the rehabilitation data collected from the third computer terminal. The fourth computer terminal 140 has a memory 142 for storing instructions and a processor 144 to execute instructions.

The third and fourth computer terminals 130, 140 may access the server 106, but not be accessible from the first and/or second computer terminals 102, 104. For example, the rehabilitation data from the third computer terminal 130 that is downloaded to the server 106 may be accessible from the fourth computer terminal 140 by the fourth user, but not accessible from the second computer terminal 104 by the second user. In this instance, the second and fourth users may both be healthcare professionals, where they separately treat the first and third users, respectively. Accordingly, the second and fourth users are only granted access to the rehabilitation data of the user that they are specifically treating.

One exemplary commercially available server system is a free-tier server hosted in Amazon Web Services (AWS). This server is running Ubuntu 16.04 LTS, on a virtualized AMD64 chip with 2 GB of RAM and a 30 GB block storage device attached. Installed on this server is a full LAMP stack including Apache 2, MySQL and PHP 7. This will support HTTPS over SSL to protect transactions between the server, the healthcare professionals (HCPs), researchers, and subjects. It will be understood that other server configurations could be used. With increased usage, more servers could be integrated to support additional load.

Data collection is facilitated by using a representational state transfer (REST) or RESTful API web services to be hosted on the server. The API will allow any supporting software to authenticate with the subject's credentials and stream performance data to the server. The data is associated with a timestamp to the microsecond and any calibrations for the game session. Performance and kinematic data collected during rehabilitation training could be securely transferred to a storage service, such as the Amazon Simple Storage Service (S3) HIPPA compliant, at the end of each training session for asynchronous analysis. Patient data can be automatically processed to produce daily progress reports.

Audio, video and screen share can be exchanged between patients and therapists via a conferencing service, such as Zoom, a cloud based, HIPPA compliant conferencing service. A patient can see and talk to a healthcare professional while the patient is playing the game. By watching patient's movement via a shared game screen, the healthcare professional can adjust the difficult of the game accordingly in real-time.

The second computer terminal 104 is coupled to the server 106 via a fiber optic or other type of communication line provided by an ISP. The second computer terminal 104 can be a desktop computer or portable device, such as a laptop, smart phone, or tablet. The terminal 104 includes a memory 120 and a processor 122. Data collected by the server 106 can be accessed from the terminal 104 and downloaded to the memory 120 for further analysis by a healthcare professional, a researcher, or a game developer. Second computer terminal 104 allows remote access to the first computer terminal 102 such that a medical professional can communicate with the patient in real-time, and adjust the difficulty of the game in response to the patient's performance. The first and second computer terminals 102, 104 need not be remotely coupled through the server 106, and may be directly electronically coupled by another means that does involve the server 106 as illustrated in FIG. 1. Exemplary means include a remote access desktop software, such as TeamViewer™ (available from TeamViewer GmbH, Goppingen, Germany).The second computer terminal 104 is capable of allowing the user to view the display of the first computer terminal 102, or at least the game window as the patient is playing a game for rehabilitation. In an embodiment, where the second computer terminal 104 is accessed by a healthcare professional who is responsible for treatment of the patient, the healthcare professional can be granted access to adjust the difficulty level of the game based on the patient's performance. For example, the second terminal 104 may allow the healthcare professional to control speed, movement sensitivity, and other difficulty parameters for the game. Exemplary types of difficulty level adjustments that can be made to the game are discussed below. In an embodiment where the healthcare professional is physically present in the patient's home, the healthcare professional may be able to adjust the settings of the game on the first computer terminal 102, or alternatively through a web-based interface using the first computer terminal 102 directly control of the game parameter. From the second computer terminal 104, games for rehabilitation can be uploaded to the server 106 and subsequently downloaded to the first computer terminal 102 by the first user. For example, a healthcare professional can use TeamViewer™ to remotely log in to patient's computer to adjust game settings. Within the game after logging into TeamViewer™, there is a menu where the therapist can make adjustments that the patient does not see.

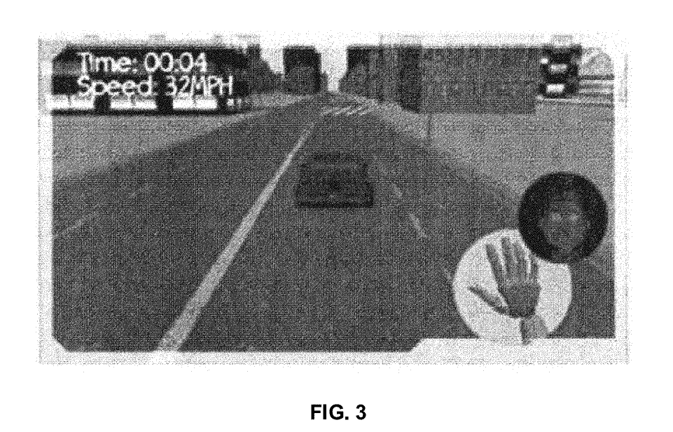

FIG. 3 depicts a screen shot of a game in accordance with the present disclosure. An embodiment of the graphical user interface (“GUI”) can include the game itself (a racing game is depicted in FIG. 3), an indicator within the game to indicate a direction the patient should move their hand if their hand strays from a position above the sensor 108, and an avatar displayed on a corner or side of the game environment to provide verbal instructions. The avatar can provide advice, instructions, praise, and encouragement to the patient. In some embodiments, in place of the avatar may be a real-time video image of a healthcare professional. The first computer terminal 102 would require a camera, either integrated or attached via USB, for a video conferencing application. One exemplary game engine is Unity Game Engine, which was used to develop the GUI and games. A skilled artisan will understand that other game engines can be utilized to achieve the same result.

In the car game depicted in FIG. 3, the patient opens and closes their hand to control the speed of the car speed. For example, making a first slows the speed of the car to make it over a speed bump. The car game is used to rehabilitate fingers and can be adjusted to change the difficulty of dynamic movements and hand postures. For example, initially the patient controls the speed of the car by opening and closing their hand. As the patient's skill level increases, the game difficulty is adjusted such that the patient has to switch lanes by pronation (turning the palm of the hand downward) or supination (turning the palm of the hand upward). The patient only advances to the next level when successfully demonstrating control of hand postures and gestures at a preceding level.

Another exemplary game is a bowling game where the patient reaches and opens their hand to apply force to the ball and knock over pins. This game targets fingers and arm. This game can be adjusted by changing the proximity of the bowling pins, e.g., adjusting the pins to be farther away increases the difficulty and moving the pins closer reduces the difficulty.

Another exemplary game is a hand flying game where the patient controls a vertical position of a plane by opening and closing the hand. By changing the vertical position of the plane, the plane intercepts floating spheres which are arranged at different vertical positions throughout the game environment. Success is measured by the number floating spheres that the patient collects. This game targets the fingers. The difficult can be adjusted by changing the speed of the plane, which causes the patient to open and close their hand faster or slower depending on the speed at which the plane is moving.

Another exemplary game is a wrist flying game where the patient controls a vertical position of the plane by changing the pitch of their hand by movement of the wrist. In this embodiment, the user controls the vertical position of the plane by changing the pitch of their hand. This game is similar to the hand flying game, and also measures achieve by the number of floating spheres collected. The difficulty of this game can also be adjusted by changing the speed of the plane.

Yet another exemplary game is an arm flying game. The object of this game is the same as the hand flying game and wrist flying game, i.e., to collect floating spheres. However, in this game, the patient controls a horizontal position of the plane by moving the arm such that the palm faces downward (pronating) or upward (supinating).

Yet another exemplary game is a fruit catching game. In this game, the user controls the location of a collection basket through horizontal arm movement to catch fruit falling from a tree. To increase the score, the patient uses pronation/supination, e.g., changes the orientation of the hand between palm up and palm down, to drop the collected fruit from the collection basket into a second basket. This game targets the wrist, elbow, and shoulder. The frequency of falling fruit decreases when player misses a certain percentage, e.g., about 25%, of the falling fruit.

Yet another exemplary game is a maze game. In this game, the patient controls the movement of the virtual avatar with their hand to guide the avatar through a maze. The patient moves their hand in the horizontal plane to guide the avatar through the maze. For example, the sensor 108 has an origin point, and can work as a virtual joystick. When the patient move their hand forward in the horizontal plane away from the origin point (in a given direction) on the sensor 108, the character runs forward in that direction until the patient returns their hand to the origin point. The same applies to any direction the hand moves in the radius around the origin point. If the patient's hand moves left, right, or a diagonal direction in the horizontal plane above the sensor 108, the character will follow this direction. The change in direction around the origin point allows the patient to move the character around corners in the maze. At increased difficulty levels, the patient needs to keep the hand steadier during the horizontal movement of the hand to guide the character along narrower paths and to stick to the path of spheres. The patient opens their hand to jump over walls. The score is defined by the number of spheres the avatar intercepts along the maze path. The user fails if the avatar deviates from the path. This game targets the elbow, shoulder, and fingers. The game difficulty can be adjusted to require more precision from the patient to guide the avatar through a path that is narrower, has sharp turns and obstacles become more frequent.

Yet another exemplary game is a soccer goalie game. In this game, the patient controls the position of the virtual hand to block the approaching soccer balls from hitting the goal. This game targets the wrist, elbow, and shoulder through horizontal and vertical motion of the patient's hand. The game difficulty can be adjusted by changing the frequency of the approaching balls.

Yet another exemplary game is a whack-a-mole game. In this game, the patient controls the position of a virtual hammer through arm movement in the horizontal plane. Rotation of the hammer, e.g., to whack a mole, is controlled by pronation or supination, e.g., turn the palm up or down. This game targets the wrist, elbow, and shoulder. The game difficulty can be adjusted by increasing the frequency of moles popping up.

Yet another exemplary game is a piano game. In this game, the patient plays a song by pressing the highlighted key with the indicated virtual finger, which corresponds to the finger of the patient. This game targets the fingers. The game difficult can be changed by adjusting the amount of individuation for the virtual fingers, e.g., increased difficulty requires the patient to move fingers more individually.

Yet another exemplary game is a breakout game. In this game, the patient controls a paddle by moving their hand in a horizontal plane to the left and to the right. The ball is directed by bouncing off the paddle and hitting the bricks above to clear the screen. This game targets the wrist, elbow and shoulder. At increased difficulty level, more balls will be provided simultaneously. The difficulty can also be adjusted by changing the paddle, so the patient has to be more precise with the paddle movement to hit the ball. The difficulty can also be adjusted by scaling the sensor so the patient has to create larger movements to move the paddle in the game.

In operation, a healthcare professional determines a game appropriate for rehabilitation of the patient's injury. The healthcare professional may then determine the appropriate level of initial difficulty level of the game based on the extent of the patient's injury. In some embodiments, the initial difficulty level may be set such that the game can be completed within a success rate of about 70% to about 85%.

Depending on the extent of the patient's injury, a support may be optionally employed to maintain the patient's hand, wrist, and/or fingers above the sensor. The patient places their arm in the support, which can be calibrated to provide the least support necessary to complete a game within the pre-determined bandwidth of success. Initially, the calibration is set at the lowest level of support necessary for the user to maintain their hand over the sensor. As the patient becomes stronger, dependency on the support can be reduced by adjusting the level of support or removing the support all together. In some embodiments, such as games that require horizontal or vertical motion of the arm, a support is not used. The patient can adjust the level of support themselves after remote consultation with the healthcare professional. Alternatively, the support may be electronically coupled to the server, and the healthcare professional may adjust the level remotely.

The patient, once the hand, wrist, and fingers are positioned above the sensor, then plays the game by moving their wrist, hand, and/or fingers such that the sensor detects the motion and translates the motion to the virtual environment of the game. If the patient is not meeting the success rate, system is able to automatically increase the game difficulty level, or the healthcare professional can adjust the game difficulty remotely. Similarly, if the patient is exceeding the success rate, system is able to automatically decrease the game difficulty level, or the healthcare professional can increase the game difficulty remotely, and/or proceed to load a new game to focus on a different aspect of the patient's rehabilitation. Exemplary parameters that can be adjusted include, but are not limited to, the intensity of practice, visual and auditory feedback, and the size and speed of the movement required interacting with task-related objects to drive individualized movement reeducation and skill development. Each game can be designed to perform specific rehabilitation tasks, which target specific rehabilitation exercises.

The motions gathered from the sensor are digitized, calibrated, and scaled to effectively measure each patient's active range of motion within the sensor's visual area, and this range is scaled to adjust into the video game virtual environment. The scaling as implemented allows for the parameters of the gaming simulations to be altered using gain scaling and adaptive algorithms, in which the rehabilitation goals and parameters can be adjusted based upon the patient's performance or other specified factors. While gain scaling is adjusted in between sessions, either by the patient or a healthcare professional, adaptive algorithms automatically adjust several parameters based on the patient's success rate in real-time.

To summarize, the present disclosure describes a rehabilitation system comprising a first computer terminal accessible by a first user, the first computer terminal having a memory for storing instructions and a processor to execute the instructions, the instructions including a game for rehabilitation of at least one of the first user's shoulder, elbow, wrist, or fingers; a sensor for detecting motion of the first user's hand, wrist, and fingers as the first user is playing the game, wherein the sensor is electrically coupled to the first computer terminal; a second computer terminal accessible by a second user, wherein the second computer terminal displays the game in real-time on the second computer terminal as the first user is playing the game on the first computer terminal, and wherein the game on the first computer terminal can be remotely adjusted from the second computer terminal; and a server electronically coupled to the first computer terminal and the second computer terminal, wherein the server acquires real-time rehabilitation data from the first computer terminal as the first user plays the game, and wherein the server permits access, on the second computer terminal, to the rehabilitation data collected.

In some embodiments, the sensor is not coupled directly to the first user's body.

In some embodiments, the server processes the rehabilitation data and generates a progress report that is accessible on the second computer terminal.

In some embodiments, a support for the patient's arm, wherein the support holds the patient's arm above the sensor and allows the patient to have free movement of the patient's wrist, hand, and fingers.

In some embodiments, the sensor further comprises at least one infrared (IR) sensor.

In some embodiments, the first and second computer terminals are electrically coupled to permit real-time communication between the first user and the second user.

In some embodiments, the real-time rehabilitation data includes motion of the first user's hand, wrist, and fingers as the first user plays the game.

In some embodiments, the server stores instructions for one or more games, wherein the instructions for the one or more games is accessible from the first and/or second computer terminals, and wherein the instructions can be copied to the memory of the first computer terminal and executed by the processor of the first computer terminal.

In some embodiments, the rehabilitation system further comprises a third computer terminal accessible by a third user, the first computer terminal having a memory for storing instructions and a processor to execute the instructions, the instructions including a game for rehabilitation of the third user's hand; and a second sensor for detecting motion of the third user's hand, wrist, and fingers as the third user is playing the game, wherein the sensor is electrically coupled to the third computer terminal, wherein the third computer terminal is electronically coupled to the server, and wherein the server acquires real-time rehabilitation data from the third computer terminal as the third user plays the game.

In some embodiments, the rehabilitation system further comprising a fourth computer terminal accessible by a fourth user, wherein the fourth computer terminal displays the game in real-time on the second computer terminal as the third user is playing the game on the third computer terminal, and wherein the game on the third computer terminal can be remotely adjusted from the fourth computer terminal, and wherein the server acquires real-time rehabilitation data from the third computer terminal as the third user plays the game, and wherein the server permits access, on the fourth computer terminal, to the rehabilitation data collected from the third computer terminal.

In some embodiments, the rehabilitation data collected from the third computer terminal is not accessible on the second computer terminal.

Also described herein is a non-transitory computer readable storage medium for a rehabilitation system, comprising storage, retrieval, modification, and linking system software which instructs at least one computer processor residing on a specialized computer system to implement a process to connect a first computer terminal accessible by a first user to a second computer terminal accessible by a second user; the first computer terminal having a memory for storing instructions and a processor to execute the instructions, the instructions including a game for rehabilitation of at least one of the first user's shoulder, elbow, wrist, or fingers; activate a sensor for detecting motion of the first user's hand, wrist, and fingers as the first user is playing the game, wherein the sensor is electrically coupled to the first computer terminal; display on the second computer terminal the game in real-time as the first user is playing the game on the first computer terminal; and adjust the difficulty level of the game on the first computer terminal by the second user of the second computer terminal.

In some embodiments, the sensor not coupled directly to the first user's body.

In some embodiments, the non-transitory computer readable storage medium further includes tracking progress of the first user utilizing the game on a centralized device or a decentralized device controlled by the second user.

In some embodiments, the sensor includes a charge-coupled device (CCD) camera or a complementary metal-oxide semiconductor (CMOS) camera.

In some embodiments, the sensor includes an infrared (IR) sensor.

Also described herein is a computer based rehabilitation method comprising connecting a first computer terminal accessible by a first user to a second computer terminal accessible by a second user, wherein the first computer terminal has a memory for storing instructions and a processor to execute the instructions, and the instructions include a game for rehabilitation of at least one of the first user's shoulder, elbow, wrist, or fingers; activating a sensor for detecting motion of the first user's hand, wrist, and fingers as the first user is playing the game, wherein the sensor is electrically coupled to the first computer terminal; displaying in real-time on the second computer terminal the game as the first user is playing the game on the first computer terminal; tracking progress of the first user utilizing the game by the second user; and adjusting the game on the first computer terminal by the second user of the second computer terminal.

In some embodiments, the sensor is not coupled directly to the first user's body.

In some embodiments the method further comprises adjusting the difficulty level of the game on the first computer terminal by the first user of the first computer terminal.

In some embodiments, adjusting the game by the second user further comprises at least one of increasing the intensity or difficulty of the game, or changing the game to a different game.

In some embodiments, the second user is a healthcare professional.

In some embodiments, the method further includes monitoring progress of the first user on a centralized device or a decentralized device controlled by the second user.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A rehabilitation system, comprising:
a first computer terminal accessible by a first user, the first computer terminal having a memory for storing instructions and a processor to execute the instructions, the instructions including a game for rehabilitation of at least one of the first user's shoulder, elbow, wrist, or fingers;
a sensor for detecting motion of the first user's hand, wrist, and fingers as the first user is playing the game, wherein the sensor is electrically coupled to the first computer terminal;
a second computer terminal accessible by a second user, wherein the second computer terminal displays the game in real-time on the second computer terminal as the first user is playing the game on the first computer terminal, and wherein the game on the first computer terminal can be remotely adjusted from the second computer terminal; and
a server electronically coupled to the first computer terminal and the second computer terminal, wherein the server acquires real-time rehabilitation data from the first computer terminal as the first user plays the game, and wherein the server permits access, on the second computer terminal, to the rehabilitation data collected.

2. The rehabilitation system of claim 1, wherein the sensor is not coupled directly to the first user's body.

3. The rehabilitation system of claim 1, wherein the server processes the rehabilitation data and generates a progress report that is accessible on the second computer terminal.

4. The rehabilitation system of claim 1, further comprising:
a support for the patient's arm, wherein the support holds the patient's arm above the sensor and allows the patient to have free movement of the patient's wrist, hand, and fingers.

5. The rehabilitation system of claim 1, wherein the sensor further comprises:
at least one infrared (IR) sensor.

6. The rehabilitation system of claim 1, wherein the first and second computer terminals are electrically coupled to permit real-time communication between the first user and the second user.

7. The rehabilitation system of claim 1, wherein the real-time rehabilitation data includes motion of the first user's hand, wrist, and fingers as the first user plays the game.

8. The rehabilitation system of claim 1, wherein the server stores instructions for one or more games, wherein the instructions for the one or more games is accessible from the first and/or second computer terminals, and wherein the instructions can be copied to the memory of the first computer terminal and executed by the processor of the first computer terminal.

9. The rehabilitation system of claim 1, further comprising:
a third computer terminal accessible by a third user, the first computer terminal having a memory for storing instructions and a processor to execute the instructions, the instructions including a game for rehabilitation of the third user's hand; and
a second sensor for detecting motion of the third user's hand, wrist, and fingers as the third user is playing the game, wherein the sensor is electrically coupled to the third computer terminal,
wherein the third computer terminal is electronically coupled to the server, and
wherein the server acquires real-time rehabilitation data from the third computer terminal as the third user plays the game.

10. The rehabilitation system of claim 9, further comprising:
a fourth computer terminal accessible by a fourth user,
wherein the fourth computer terminal displays the game in real-time on the second computer terminal as the third user is playing the game on the third computer terminal, and wherein the game on the third computer terminal can be remotely adjusted from the fourth computer terminal, and
wherein the server acquires real-time rehabilitation data from the third computer terminal as the third user plays the game, and wherein the server permits access, on the fourth computer terminal, to the rehabilitation data collected from the third computer terminal.

11. The rehabilitation system of claim 10, wherein the rehabilitation data collected from the third computer terminal is not accessible on the second computer terminal.

12. A non-transitory computer readable storage medium for a rehabilitation system, comprising storage, retrieval, modification, and linking system software which instructs at least one computer processor residing on a specialized computer system to implement a process to:
connect a first computer terminal accessible by a first user to a second computer terminal accessible by a second user;
the first computer terminal having a memory for storing instructions and a processor to execute the instructions, the instructions including a game for rehabilitation of at least one of the first user's shoulder, elbow, wrist, or fingers;
activate a sensor for detecting motion of the first user's hand, wrist, and fingers as the first user is playing the game, wherein the sensor is electrically coupled to the first computer terminal;
display on the second computer terminal the game in real-time as the first user is playing the game on the first computer terminal; and
adjust the difficulty level of the game on the first computer terminal by the second user of the second computer terminal.

13. The non-transitory computer readable storage medium for a rehabilitation system of claim 12, wherein the sensor is not coupled directly to the first user's body.

14. The non-transitory computer readable storage medium for a rehabilitation system of claim 12, further includes:
tracking progress of the first user utilizing the game on a centralized device or a decentralized device controlled by the second user.

15. The non-transitory computer readable storage medium for a rehabilitation system of claim 12, wherein the sensor includes a charge-coupled device (CCD) camera or a complementary metal-oxide semiconductor (CMOS) camera.

16. The non-transitory computer readable storage medium for a rehabilitation system of claim 12, wherein the sensor includes an infrared (IR) sensor.

17. A computer based rehabilitation method, comprising:
connecting a first computer terminal accessible by a first user to a second computer terminal accessible by a second user, wherein the first computer terminal has a memory for storing instructions and a processor to execute the instructions, and the instructions include a game for rehabilitation of at least one of the first user's shoulder, elbow, wrist, or fingers;
activating a sensor for detecting motion of the first user's hand, wrist, and fingers as the first user is playing the game, wherein the sensor is electrically coupled to the first computer terminal;
displaying in real-time on the second computer terminal the game as the first user is playing the game on the first computer terminal;
tracking progress of the first user utilizing the game by the second user; and
adjusting the game on the first computer terminal by the second user of the second computer terminal.

18. The computer based rehabilitation method of claim 17, wherein the sensor is not coupled directly to the first user's body.

19. The computer based rehabilitation method of claim 17, further comprising:
adjusting the difficulty level of the game on the first computer terminal by the first user of the first computer terminal.

20. The computer based rehabilitation method of claim 17, wherein adjusting the game by the second user further comprises at least one of increasing the intensity or difficulty of the game, or changing the game to a different game.
